(19) United States
(12) Patent Application Publication
(10) Pub. No.: US 2024/0425455 A1
(43) Pub. Date: Dec. 26, 2024

(54) METHOD FOR SELECTIVELY AND CONTINUOUSLY PRODUCING 2-METHYLPYRIDINE AND DIPHENYLAMINE FROM ANILINE

(71) Applicants: CHINA PETROLEUM & CHEMICAL CORPORATION, Beijing (CN); SINOPEC DALIAN RESEARCH INSTITUTE OF PETROLEUM AND PETROCHEMICALS CO., LTD., Dalian, Liaoning (CN)

(72) Inventors: Haomeng LI, Dalian, Liaoning (CN); Xiangyu ZHAO, Dalian, Liaoning (CN); Libo WANG, Dalian, Liaoning (CN); Wenbo QI, Dalian, Liaoning (CN); Zhenyu WANG, Dalian, Liaoning (CN); Lanpeng LI, Dalian, Liaoning (CN); Fubin AI, Dalian, Liaoning (CN)

(21) Appl. No.: 18/705,968

(22) PCT Filed: Oct. 19, 2022
(86) PCT No.: PCT/CN2022/126220

§ 371 Date: Apr. 29, 2024

(30) Foreign Application Priority Data
Oct. 31, 2021 (CN) 202111278790.6

(51) Int. Cl. C07D 213/127; B01J 29/70; C07C 209/02

(52) U.S. Cl. CPC C07D 213/127; B01J 29/7007; C07C 209/02; C07C 2529/04

(57) ABSTRACT

The present invention relates to the field of fine chemical industry. Specifically disclosed are a method for synthesizing 2-methylpyridine from aniline, and a method for selectively and continuously producing 2-methylpyridine and diphenylamine from aniline. The method comprises: in a hydrogen-containing atmosphere, enabling aniline and a catalyst to undergo a contact reaction at a temperature of 100-400° C., wherein the catalyst is β zeolite carrying a metal component, and the metal component comprises an active metal component selected from at least one of W, Mo, Ni, and Co; controlling the temperature of the contact reaction to be 100-240° C. to obtain a product mainly consisting of 2-methylpyridine; and controlling the temperature of the contact reaction to be 260-400° C. to obtain a product mainly consisting of diphenylamine.

### CROSS-REFERENCE TO RELATED APPLICATIONS

The application claims the benefit of Chinese patent application No. “202111278790.6”, filed on Oct. 31, 2021, the content of which is specifically and entirely incorporated herein by reference.

### TECHNICAL FIELD

The invention belongs to the technical field of the fine chemical industry and particularly relates to a method for synthesizing 2-methylpyridine from aniline and a method for selectively and continuously producing 2-methylpyridine and diphenylamine from aniline.

### BACKGROUND ART

2-methylpyridine, represented by the molecular formula C₆H₇N, also called α-methylpyridine or α-picoline, is one kind of pyridine base, it is a colorless oily liquid at room temperature and has a strong unpleasant odor, it has a freezing point of −38.9° C. and a boiling point of 129.5° C. as well as strong toxicity, and is mutually soluble with solvents such as acetone, ethanol, ether, and water. 2-methylpyridine is an important chemical intermediate and an important starting material in the fine chemical industry, can be used for producing long-acting sulfanilamide, pesticide intermediates, and feedstuff intermediates, and can also be used for synthesizing special resin vinyl pyridine and 2-vinyl pyridine intermediates. The used amount of 2-methylpyridine in China increases at a rate of about 25% per year, and thus the domestic market potential of 2-methylpyridine is tremendous.

The production methods of pyridine base in the industrial field mainly comprise a coal tar separation method and a chemical synthesis method. The coal tar separation method has the defects such as serious pollution, a few product types, high investment and costs, poor quality, high energy consumption, and the yield of the product is low and can hardly be improved. The chemical synthesis method usually adopts an ammonium aldehyde condensation method and uses aldehyde, ketone, and ammonia as starting materials to react and synthesize the pyridine base. However, the products of the ammonium aldehyde condensation method include 3-methylpyridine, 2-methylpyridine, 4-methylpyridine, and the like, the method has the problems such as difficulty in separating subsequent products.

CN105384683A discloses a method of separating byproducts 2-methylpyridine and 4-methylpyridine from the process of synthesizing diphenylamine with aniline, the steps are as follows: the aniline flows through a fixed bed reactor containing a molecular sieve catalyst, the reactor temperature and pressure are controlled; by the condensation reaction, the aniline is converted into a mixture reaction solution comprising diphenylamine, ammonia, and byproducts such as 2-methylpyridine, 4-methylpyridine, water, acridine, and 4-aminobiphenyl, the low boiling point mixed fraction is obtained by multistage distillation of the mixture reaction solution, the low boiling point mixed fraction is dehydrated, the dehydrated mixture is dried, the dried mixture is distilled to obtain 2-methylpyridine and 4-methylpyridine according to different boiling points. The essence of the method is a process of separating byproducts 2-methylpyridine and 4-methylpyridine from the traditional process of synthesizing diphenylamine with aniline. Therefore, given that the 2-methylpyridine is a byproduct in the reaction process, the yield is low, and the method is not suitable for large-scale production.

CA1190928A discloses a method for producing α-picoline from aniline, wherein the starting material is passed over an acid zeolite catalyst in the presence of inert gas (e.g., nitrogen gas and helium gas) at the temperature range of 200-650° C. to prepare a product containing alpha-picoline, the reaction pressure is from atmospheric up to 25,000 kPa, usually at 200 kPa, and the space velocity of aniline is preferably 0.2-5 WHSV. The acidic zeolite is preferably in the hydrogen form and includes beta-zeolite having a silica: alumina ratio of 10-100, up to 150. Wherein Example 1 uses the pure HZSM-5, the conversion rate of aniline is 13.1% and the selectivity of α-picoline is 51.6% under the conditions consisting of the temperature of 510° C., the pressure of 2,860 kPa, ammonia gas accounts for 1.5 mol % of aniline, and the space velocity of nitrogen gas is 100 GHSV.

“The zeolite-catalysed isomerization of aniline to α-picoline [J]. Applied catalysis A: General 172(1998)285-294” discloses a method for preparing α-picoline by means of an aniline rearrangement reaction, the method uses a molecular sieve, preferably Ga-MFI molecular sieve (better than ZSM-5), as the catalyst, and performed under the conditions consisting of a temperature of 673K, a total pressure of 75 bar, and an ammonia partial pressure of 5-60 bar, preferably 20-25 bar. Wherein the selectivity of the α-picoline can reach 85% or more, but the conversion rate is only 3.7%.

### SUMMARY OF THE INVENTION

The invention aims to overcome the defects in the prior art that the method for preparing 2-methylpyridine by using the aniline rearrangement reaction has a low conversion rate of aniline or the low selectivity of 2-methyl pyridine and provides a method for selectively producing diphenylamine and 2-methylpyridine based on the existing device for producing diphenylamine from aniline. The method of the invention can conveniently and effectively realize the switching between the diphenylamine product and the 2-methylpyridine product by controlling the temperature, the reaction conditions are mild, the yield of 2-methylpyridine is high, the reaction reactivity is desirable, thus the method has excellent industrial application prospect.

The inventors of the present invention have discovered that both the reaction for generating 2-methylpyridine by isomeric rearrangement of aniline and the reaction for preparing diphenylamine by condensation of aniline are influenced by two factors of the catalyst and the reaction temperature. The invention uses a β zeolite catalyst carrying an active metal component selected from at least one of W, Mo, Ni, and Co, the reaction route of aniline can be switched between 2-methylpyridine and diphenylamine due to the change of the catalytic activity center. In addition, the lower reaction temperature is more beneficial to the synthesis of 2-methylpyridine. Based on the finding, the present invention proposes a method for producing 2-methylpyridine and a method for selectively and continuously producing diphenylamine and 2-methylpyridine.

The first aspect of the invention provides a method for selectively and continuously producing 2-methylpyridine and diphenylamine from aniline, the method comprises in a hydrogen-containing atmosphere, enabling an aniline and a catalyst to undergo a contact reaction at a temperature of 100-400° C., wherein the catalyst is a β zeolite carrying a metal component, and the metal component comprises an active metal component selected from at least one of W, Mo, Ni, and Co; controlling the temperature of the contact reaction to be 100-240° C. to obtain a product mainly consisting of 2-methylpyridine; and controlling the temperature of the contact reaction to be 260-400° C. to obtain a product mainly consisting of diphenylamine.

The second aspect of the present invention provides a method for synthesizing 2-methylpyridine from aniline, wherein the method comprises carrying out a contact reaction between aniline and a catalyst under a hydrogen-containing atmosphere and a condition for isomeric rearrangement reaction condition, the catalyst is a β zeolite carrying a metal component comprising an active metal component selected from at least one of W, Mo, Ni, and Co, and the temperature of the contact reaction is within the range of 100-240° C.

The method in the invention is used for producing 2-methylpyridine, it has the advantages of a high conversion rate of aniline and high selectivity of the target product 2-methylpyridine. The possible reasons may be deduced as follows: under a hydrogen-containing atmosphere, the reaction mechanism is greatly changed due to the selection of a β zeolite catalyst containing at least one active metal of W, Mo, Ni, and Co, thermodynamic factors become one of the factors influencing the selectivity of 2-methy pyridine, the reaction of generating one diphenylamine molecule by condensing two aniline molecules is the main reaction at a high temperature, and by reducing the temperature, the reaction is switched to the main reaction of generating 2-methy pyridine by isomeric rearrangement of the aniline molecules. Therefore, the method of the invention can realize the purpose of producing 2-methylpyridine by using aniline as a starting material and selectively producing diphenylamine and 2-methylpyridine on the existing process device of diphenylamine.

In the prior art, a large amount of ammonia gas is generally introduced for synthesizing pyridine base compounds. The presence of ammonia gas will occupy the acidic site of the catalyst, resulting in reduced catalyst activity. At the same time, the high temperature and high pressure also limit the industrial application of the process. The method provided by the invention only needs to introduce a small amount of hydrogen gas without introducing a large amount of ammonia gas, and the reaction conditions are mild, only the aniline is used as the reaction starting material, the high-purity 2-methylpyridine and high-purity diphenylamine can be obtained through the subsequent distillation operation.

### BRIEF DESCRIPTION OF THE DRAWINGS

FIG. 1 is a graph showing the relationship between the product distribution of a method for selecting and continuously producing 2-methylpyridine and diphenylamine from amine provided by the present invention along with the change of temperature.

### DESCRIPTION OF THE PREFERRED EMBODIMENT

The terminals and any value of the ranges disclosed herein are not limited to the precise ranges or values, such ranges or values shall be comprehended as comprising the values adjacent to the ranges or values. As for numerical ranges, the endpoint values of the various ranges, the endpoint values and the individual point value of the various ranges, and the individual point values may be combined with one another to produce one or more new numerical ranges, which should be deemed have been specifically disclosed herein.

The invention provides a method for synthesizing 2-methylpyridine from aniline, the method comprises carrying out a contact reaction between aniline and a catalyst under a hydrogen-containing atmosphere and a condition for isomeric rearrangement reaction, the catalyst is a β zeolite carrying a metal component comprising an active metal component selected from at least one of W, Mo, Ni, and Co, and the temperature of the contact reaction is within the range of 100-240° C.

The invention unexpectedly improves the selectivity of 2-methylpyridine while obtaining the high conversion rate of aniline by using a β zeolite carrying an active metal component selected from at least one of W, Mo, Ni, and Co in a hydrogen-containing atmosphere, and controlling the reaction temperature to be lower than the temperature generally required by the reaction for synthesizing diphenylamine from aniline. Moreover, the method can realize the selective production of diphenylamine and 2-methylpyridine by adjusting the reaction temperature and using the same set of devices with the same catalyst.

According to a preferred embodiment of the invention, the active metal component in terms of metal element is contained in an amount of 0.5-5 wt. %, preferably 0.5-3 wt. %, based on the total amount of the catalyst.

Preferably, the metal component further comprises an auxiliary metal component selected from at least one of Li, Na, K, Mg, and Ca, the auxiliary metal component in terms of an oxidation state is contained in an amount of 0-6.5 wt. %, preferably 0.5-5.5 wt. %, based on the total amount of the catalyst.

By adding the auxiliary metal component, both the acid distribution on the surface of the catalyst and the pore structure of the catalyst can be improved, and both the conversion rate of aniline and the selectivity of a target product are further increased.

Although the mechanism is not clear, the present inventors have discovered that the above-mentioned effect of producing diphenylamine or 2-methylpyridine as the main product by adjusting the temperature can only be obtained by loading the above-mentioned metal component on a β zeolite under a hydrogen-containing atmosphere, the above-mentioned effect cannot be achieved by using other molecular sieves such as ZSM-5.

Preferably, the β zeolite has a Si/Al molar ratio within the range of 25-300, preferably within the range of 60-220.

Preferably, the catalyst has a specific surface area of 400-700 m²/g, more preferably 450-650 m²/g; a pore volume of 0.25-0.6 mL/g, more preferably 0.4-0.55 mL/g; an average pore diameter of 1.5-5 nm, more preferably 2-4 nm; and a particle size preferably less than 9 nm.

The catalyst may further comprise an alumina binder, thereby obtaining a shaped body of the catalyst.

According to a preferred embodiment of the invention, the catalyst comprises 50-85 wt. % of zeolite, 0.5-5 wt. % of an active metal component in terms of the metal element, 0-6.5 wt. % of an auxiliary metal component in terms of oxidation state, and 10-45 wt. % of an alumina binder, based on the total amount of the catalyst. Preferably, the catalyst comprises 60-85 wt. % of zeolite, 0.5-3 wt. % of an active metal component in terms of the metal element, 0.5-5.5 wt. % of an auxiliary metal component in terms of oxidation state, and 15-34.5 wt. % of an alumina binder, based on the total amount of the catalyst.

Preferably, the catalyst is consisting of strip-shaped or spherical particles; when the catalyst is consisting of strip-shaped particles, the cross-section of the catalyst can be cylindrical, cloverleaf-shaped or four-leaf clover-shaped, and the cross-section width of the strip-shaped particle is within the range of 0.5-3.0 mm, preferably within the range of 1.0-2.0 mm; when the catalyst is consisting of spherical particles, the particles have a diameter within the range of 0.5-5.0 mm, preferably within the range of 1.0-3.0 mm.

The above-mentioned catalysts can be prepared with the methods generally known by those skilled in the art or are commercially available. For example, the catalyst of the present invention can be prepared with the following method: (1) contacting Hβ zeolite with an aqueous nitrate solution of an auxiliary metal component in an equivalent-volume impregnation mode; (2) filtering, washing, and drying the mixture obtained in step (1); (3) fully kneading the modified Hβ zeolite obtained in step (2), an alumina binder and processing auxiliary agents (e.g., an extrusion auxiliary agent and a peptizing agent), molding, and then drying and roasting; (4) adding NaBH₄ into the carrier obtained in step (3) and an aqueous nitrate solution of an active metal component for reflux reduction, filtering, and drying in vacuum to prepare the catalyst.

Wherein the contact temperature in step (1) is within the range of 80-100° C., and the contact time is 3-6 h; drying in step (2) is performed at 60-120° C. for 6-12 h; drying in step (3) is performed at 60-120° C. for 6-12 h; roasting is performed at the temperature of 450-550° C. for 4-16 h; the reflux reduction in step (4) is implemented at the temperature of 80-100° C. for 6-8 h, and the vacuum drying is then carried out at the temperature of 80-100° C. for 6-12 h.

By using the above catalyst, the invention can obtain a high yield of 2-methylpyridine at a lower temperature under a hydrogen-containing atmosphere rather than the traditional ammonia gas atmosphere. Therefore, the contact reaction is preferably performed under the circumstance without introducing ammonia gas. The hydrogen-containing atmosphere refers to the concentration of hydrogen gas within the range of 10-100 vol. %.

Further, the temperature of the contact reaction is within the range of 100-240° C. The specific reaction temperature may be 100° C., 110° C., 120° C., 130° C., 140° C., 150° C., 160° C., 170° C., 180° C., 190° C., 199° C., 200° C., 210° C., 220° C., 230° C., 240° C., and any value within the range consisting of any two of the point values. The preferable reaction temperature may be within the range of 130-180° C., the specific reaction temperature may be 130° C., 140° C., 150° C., 160° C., 170° C., 180° C., and any value within the range consisting of any two of the point values.

The method of the present invention may be carried out continuously or intermittently and is preferably carried out continuously. The intermittent operation is usually a kettle-type operation, and the target product can be obtained by adding the aniline, and the catalyst into a reaction kettle and carrying out the reaction under appropriate conditions. The continuous operation generally refers to the aniline is reacted through a fixed bed reactor containing the catalyst.

The present inventors have found that the optimum reaction temperature and pressure are slightly different between the continuous reaction and the intermittent reaction. When the intermittent reaction is adopted, the temperature of the contact reaction is preferably within the range of 130-180° C.; the pressure of the contact reaction is preferably within the range of 2-4 MPa, and the reaction time is preferably within the range of 4-8 h; the used amount of the catalyst preferably accounts for 1-4 wt. % of the used amount of the aniline. When the continuous reaction is adopted, the temperature of the contact reaction is preferably within the range of 160-240° C.; the pressure of the contact reaction is preferably within the range of 1.5-3 MPa, and the liquid hourly mass space velocity of the aniline is preferably within the range of 0.5-3 h⁻¹, further preferably 1-1.5 h⁻¹.

The continuous operation is preferably carried out by feeding the aniline into the bottom of the fixed bed reactor and passing through a catalyst bed of the fixed bed reactor comprising the catalyst.

The present inventors have also found that the reaction for generating 2-methylpyridine by isomeric rearrangement of aniline and the reaction for preparing diphenylamine by condensation of aniline are influenced by the two factors of catalyst and reaction temperature. Use of the metal-loaded β zeolite catalyst of the present invention, the reaction route of aniline can be switched between 2-methylpyridine and diphenylamine due to the change of the catalytic activity center. Moreover, the low reaction temperature is more favorable for synthesizing 2-methylpyridine, and the high temperature is conducive to the generation of diphenylamine.

Therefore, the invention also provides a method for selectively and continuously producing 2-methylpyridine and diphenylamine from aniline, the method comprises: in a hydrogen-containing atmosphere, enabling an aniline and a catalyst to undergo a contact reaction at a temperature of 100-400° C., wherein the catalyst is zeolite carrying a metal component, and the metal component comprises an active metal component selected from at least one of W, Mo, Ni, and Co, controlling the temperature of the contact reaction to be 100-240° C. to obtain a product mainly consisting of 2-methylpyridine; and controlling the temperature of the contact reaction to be 260-400° C. to obtain a product mainly consisting of diphenylamine.

Preferably, when a product mainly containing 2-methylpyridine is obtained, the temperature of the contact reaction is within the range of 160-240° C., the pressure of the contact reaction is within the range of 1.5-3 MPa, and the liquid hourly mass space velocity of the aniline is preferably within the range of 0.5-3 h⁻¹; when a product mainly consisting of diphenylamine is obtained, the temperature of the contact reaction within the range of 280-360° C., the pressure of the contact reaction is within the range of 1.5-3 MPa, and the liquid hourly mass space velocity of the aniline is preferably within the range of 0.3-1.5 h⁻¹.

In the invention, the product mainly containing 2-methylpyridine refers to that the selectivity of 2-methylpyridine is not less than 40%, and the product mainly containing diphenylamine refers to the selectivity of diphenylamine is not less than 40%.

The catalyst is described above and will not be repeatedly described herein.

The conditions for synthesizing diphenylamine with aniline in the invention comprise: the temperature of the contact reaction is preferably within the range of 280-360° C., the pressure of the contact reaction is preferably within the range of 1.5-3 MPa, and the liquid hourly mass space velocity of the aniline is preferably within the range of 0.3-1.5 h⁻¹, further preferably 0.5-1 h⁻¹.

In the Examples and Comparative Examples, the calculation methods of the conversion rate of aniline, the selectivity and yield of 2-methylpyridine, and the selectivity and yield of diphenylamine are as follows:

The conversion rate of aniline=(mole number of aniline in starting material before reaction−mole number of aniline in the product)/mole number of aniline in starting material before reaction×100%;

The yield of 2-methylpyridine=mole number of 2-methylpyridine in the product/mole number of all the aniline in starting material before reaction theoretically converted into 2-methylpyridine;

The selectivity of 2-methylpyridine=(mole number of 2-methylpyridine in the product)/(mole number of aniline in starting material before reaction-mole number of aniline in the product)×100%;

The yield of diphenylamine=molar number of diphenylamine in the product/molar number of all the aniline in the starting material before the reaction theoretically converted into diphenylamine;

The selectivity of diphenylamine=2×(mole number of diphenylamine in the product)/(mole number of aniline in the starting material before reaction-mole number of aniline in the product)×100%.

The contents of aniline, diphenylamine, and 2-methylpyridine were measured according to gas chromatography.

The alumina binder in use was a commercially available β-alumina, and the zeolite in use was a commercially available Hβ zeolite having a specific surface area of 620 m²/g and a particle size less than 9 nm.

The catalysts used in the Examples and Comparative Examples were prepared with the method of loading auxiliary metal component and active metal component by the saturated impregnation method, and the concrete method was as follows: (1) Hβ zeolite contacted with an aqueous nitrate solution of an auxiliary metal component in an equivalent-volume impregnation mode; (2) the mixture obtained in step (1) was subjected to filtering, washing, and drying; (3) the modified Hβ zeolite obtained in step (2), an alumina binder (e.g., Pseudo-Boehmite) and processing auxiliary (e.g., an extrusion auxiliary and a peptizing agent) were subjected to fully kneading, molding, and then drying and roasting; (4) NaBH₄ was added into the carrier obtained in step (3) and an aqueous nitrate solution of an active metal component for reflux reduction, then subjected to filtering, and drying in vacuum to prepare the spherical catalyst with the particle diameter of 3 mm. Wherein the contact temperature in step (1) was 90° C., and the contact time was 4 h; drying in step (2) was performed at 80° C. for 10 h; drying in step (3) was performed at 80° C. for 10 h; roasting was performed at the temperature of 550° C. for 10h; the reflux reduction in step (4) was implemented at the temperature of 100° C. for 8 h, and the vacuum drying was then carried out at the temperature of 100° C. for 10 h.

The properties of the catalysts were listed in Table 1.

TABLE 1
Catalysts

| | | Catalysts | | | | | | | | |
|---|---|---|---|---|---|---|---|---|---|---|
| | | A | B | C | D | E | F | G | H | I |
| Active | Types | Ni | Ni | Ni | Mo | Ni | Co | Ni | — | Ni |
| metal | Content | 0.5 | 1.5 | 2 | 1.5 | 5 | 1.5 | 1.5 | — | 1.5 |
| | (wt. %) | | | | | | | | | |
| Hβ zeolite (wt. %) | | 60 | 82 | 80 | 80 | 75 | 80 | 82 | 82 | ZSM-5 |
| Si/Al ratio of Hβ | | 60 | 60 | 200 | 60 | 60 | 60 | 60 | 60 | — |
| zeolite | | | | | | | | | | |
| Auxiliary | Types | K | Mg | K | Na | Ca | Na | — | Mg | Mg |
| metal | Content in | 5.2 | 1 | 0.5 | 3 | 2 | 3 | — | 1 | 1 |
| | terms of | | | | | | | | | |
| | oxidation | | | | | | | | | |
| | state | | | | | | | | | |
| | (wt. %) | | | | | | | | | |

| | | | | | | | | | |
|---|---|---|---|---|---|---|---|---|---|
| Alumina | Balance | Balance | Balance | Balance | Balance | Balance | Balance | Balance | Balance |

### Examples 1-8

A kettle-type reactor was selected, the reaction was carried out in a hydrogen atmosphere, the used amount of the catalyst was 1 g, the added amount of the aniline was 50 g, and the reaction conditions were as shown in Table 2 below.

### Comparative Examples 1-2

The conversion reaction of aniline was carried out according to the method of Example 1, except that the reaction conditions were as shown in Table 2 below.

### Comparative Example 3

The conversion reaction of aniline was carried out according to the method of Example 1, except that β zeolite (metal component was not loaded) was used as the catalyst, and other reaction conditions were shown in Table 2 below.

### Comparative Example 4

The production of 2-methylpyridine from aniline was performed according to the method in Example 1 of CA1190928A as follows: pure HZSM-5 was used as the catalyst for converting the aniline into the 2-methylpyridine under the conditions consisting of a temperature of 510° C., a pressure of 2.9 MPa, molar ratio of the aniline to ammonia of 1:8, and the mass space velocity of 1.0 h⁻¹.

TABLE 2

| | | | Reaction | Reaction | | Conversion | The yield of | The selectivity of |
|---|---|---|---|---|---|---|---|---|
| | | | temperature | pressure | Reaction | rate of aniline | 2-methylpyridine | 2-methylpyridine |
| | | Catalysts | (° C.) | (MPa) | time (h) | (mol %) | (mol %) | (mol %) |
| Examples | 1 | A | 130 | 4 | 6 | 40.2 | 24.4 | 60.7 |
| | 2 | B | 180 | 4 | 6 | 45.4 | 25.7 | 56.6 |
| | 3 | C | 200 | 3 | 6 | 46.2 | 20.9 | 45.3 |
| | 4 | D | 100 | 2 | 4 | 36.7 | 22.5 | 61.3 |
| | 5 | E | 130 | 4 | 6 | 37.6 | 21.6 | 57.4 |
| | 6 | F | 150 | 2 | 8 | 42.4 | 24.6 | 58.1 |
| | 7 | B | 220 | 4 | 6 | 47.4 | 23.4 | 49.3 |
| | 8 | G | 180 | 4 | 6 | 33.2 | 16.0 | 48.3 |
| Comparative | 1 | I | 200 | 3 | 6 | 15.5 | 3.9 | 25.3 |
| Examples | 2 | A | 260 | 4 | 6 | 46.3 | 14.2 | 30.7 |
| | 3 | H | 180 | 4 | 6 | 20.6 | 0.8 | 3.9 |
| | 4 | ZSM-5 | 510 | 2.9 | 1 | 13.7 | 7.1 | 51.6 |

### Examples 9-16

A fixed bed reactor was selected, and the aniline passed through a catalyst bed in the lower feeding mode, the reaction was firstly carried out for 48 hours under the condition suitable for generating 2-methylpyridine in a hydrogen atmosphere, then the reaction was continued for 48 hours by switching to the condition suitable for generating diphenylamine at the temperature rise rate of 30° C./hour, and the measured product distribution was shown in the following Table 3.

TABLE 3

| Examples | | | | | | | |
|---|---|---|---|---|---|---|---|
| 9 | 10 | 11 | 12 | 13 | 14 | 15 | 16 |

Before 48 h

| | | | | | | | | |
|---|---|---|---|---|---|---|---|---|
| Catalysts | B | B | C | D | C | C | A | G |
| Reaction temperature | 160 | 220 | 200 | 200 | 240 | 260 | 160 | 160 |
| (° C.) | | | | | | | | |
| Reaction pressure (MPa) | 1.5 | 3 | 2 | 3 | 2 | 2 | 1.5 | 1.5 |
| Liquid hourly mass space | 1.5 | 1.5 | 1 | 1.5 | 1 | 1 | 1.5 | 1.5 |
| velocity (h-1) | | | | | | | | |
| The conversion rate of | 33.5 | 34.7 | 38.1 | 37.7 | 38.3 | 38.9 | 30.6 | 26.7 |
| aniline (mol %) | | | | | | | | |
| The yield of | 22.6 | 21.8 | 24.6 | 22.1 | 21 | 18.4 | 20.3 | 16.9 |
| 2-methylpyridine (mol %) | | | | | | | | |
| Selectivity of | 67.6 | 62.9 | 64.5 | 58.6 | 54.8 | 47.4 | 66.3 | 63.4 |
| 2-methylpyridine (mol %) | | | | | | | | |

48-96 hours

| | | | | | | | | |
|---|---|---|---|---|---|---|---|---|
| catalyst | B | B | C | D | C | C | A | G |
| Reaction temperature | 280 | 315 | 360 | 340 | 295 | 340 | 280 | 280 |
| (° C.) | | | | | | | | |
| Reaction pressure (MPa) | 2 | 1.5 | 3 | 2 | 1.5 | 2 | 2 | 2 |
| Liquid hourly mass space | 0.5 | 1 | 0.8 | 1 | 0.9 | 1 | 0.5 | 0.5 |
| velocity (h⁻¹) | | | | | | | | |
| The conversion rate of | 39.2 | 39.8 | 42.1 | 41.3 | 38.1 | 40.7 | 37.8 | 33.2 |
| aniline (mol %) | | | | | | | | |
| Selectivity of | 49.4 | 50.3 | 56.6 | 51.2 | 53.3 | 55.7 | 53.5 | 43.6 |
| diphenylamine (mol %) | | | | | | | | |

### Example 17

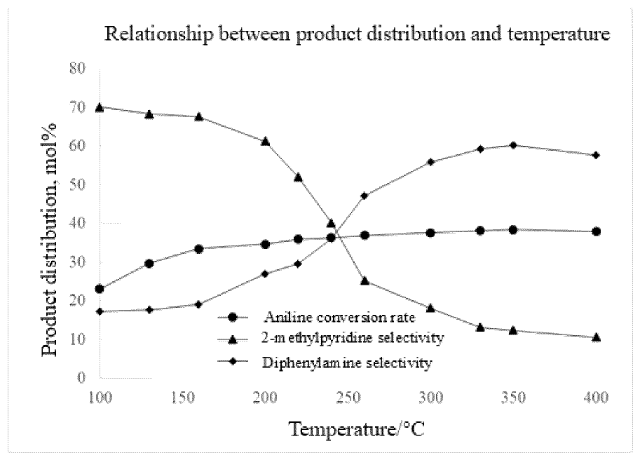

Diphenylamine and 2-methylpyridine were produced from aniline according to the method of Example 9, in order to examine the influence of temperature on the distribution of products, and after each sampling, the temperature was raised at a temperature-rise rate of 30° C./hour until the next temperature, and the graph of the product distribution change along with the temperature was shown in FIG. 1.

As can be seen from the results of Table 2 and FIG. 1, the method for preparing 2-methylpyridine provided by the invention can obtain the high conversion rate of aniline and high selectivity of 2-methylpyridine at a low reaction temperature.

It can be derived from the results of Table 3 and FIG. 1 that the method for selectively and continuously producing diphenylamine and 2-methylpyridine provided by the invention can conveniently produce different target products diphenylamine and 2-methylpyridine by adjusting the reaction temperature, pressure, and other process parameters on a set of apparatus.

1. A method for selectively and continuously producing 2-methylpyridine and diphenylamine from aniline, the method comprises: in a hydrogen-containing atmosphere, enabling an aniline and a catalyst to undergo a contact reaction at a temperature of 100-400° C., wherein the catalyst is a β zeolite carrying a metal component, and the metal component comprises an active metal component selected from at least one of W, Mo, Ni, and Co; controlling the temperature of the contact reaction to be 100-240° C. to obtain a product mainly consisting of 2-methylpyridine, and controlling the temperature of the contact reaction to be 260-400° C. to obtain a product mainly consisting of diphenylamine.

2. The method according to claim 1, wherein the active metal component in terms of metal element is contained in an amount of 0.5-5 wt. %, based on the total amount of the catalyst.

3. The method according to claim 1, wherein the metal component further comprises an auxiliary metal component selected from at least one of Li, Na, K, Mg, and Ca, the auxiliary metal component in terms of an oxidation state is contained in an amount of 0-6.5 wt. %, based on the total amount of the catalyst;
and/or, the active metal component in terms of metal element is contained in an amount of 0.5-3 wt. %, based on the total amount of the catalyst.

4. The method according to claim 1, wherein the catalyst further comprises an alumina binder, the catalyst comprises 50-85 wt. % of a β zeolite, 0.5-5 wt. % of an active metal component in terms of metal element, 0-6.5 wt. % of an auxiliary metal component in terms of oxidation state, and 10-45 wt. % of an alumina binder, based on the total amount of the catalyst;
and/or, the metal component further comprises an auxiliary metal component selected from at least one of Li, Na, K, Mg, and Ca, the auxiliary metal component in terms of an oxidation state is contained in an amount of 0.5-5.5 wt. %, based on the total amount of the catalyst.

5. The method according to claim 4, wherein the catalyst comprises 60-85 wt. % of a β zeolite, 0.5-3 wt. % of an active metal component in terms of the metal element, 0.5-5.5 wt. % of an auxiliary metal component in terms of oxidation state, and 15-34.5 wt. % of an alumina binder, based on the total amount of the catalyst.

6. The method according to claim 1, wherein the pressure of the contact reaction is within a range of 1.5-4 MPa, the contact reaction is carried out under a hydrogen atmosphere.

7. The method according to claim 1, wherein the contact reaction is performed under the circumstance without introducing ammonia gas.

8. The method according to claim 1, when a product mainly consisting of 2-methylpyridine is obtained, the temperature of the contact reaction is within the range of 160-240° C., the pressure of the contact reaction is within the range of 1.5-3 MPa; or
when a product mainly consisting of diphenylamine is obtained, the temperature of the contact reaction is within the range of 280-360° C., the pressure of the contact reaction is within the range of 1.5-3 MPa.

9. The method according to claim 8, wherein the aniline is reacted through a fixed bed reactor containing the catalyst;
and/or, when a product mainly consisting of 2-methylpyridine is obtained, the liquid hourly mass space velocity of the aniline is within the range of 0.5-3 h⁻¹.
and/or, when a product mainly consisting of diphenylamine is obtained, the liquid hourly mass space velocity of the aniline is within the range of 0.3-1.5 h⁻¹.

10. A method for synthesizing 2-methylpyridine from aniline, wherein the method comprises carrying out a contact reaction between an aniline and a catalyst under a hydrogen-containing atmosphere and a condition for isomeric rearrangement reaction, the catalyst is a β zeolite carrying a metal component comprising an active metal component selected from at least one of W, Mo, Ni, and Co, and the temperature of the contact reaction is within the range of 100-240° C.

11. The method according to claim 10, wherein the active metal component in terms of metal element is contained in an amount of 0.5-5 wt. %, based on the total amount of the catalyst.

12. The method according to claim 10, wherein the metal component further comprises an auxiliary metal component selected from at least one of Li, Na, K, Mg, and Ca, the auxiliary metal component in terms of an oxidation state is contained in an amount of 0-6.5 wt. %, based on the total amount of the catalyst;
and/or, the active metal component in terms of metal element is contained in an amount of 0.5-3 wt. %, based on the total amount of the catalyst.

13. The method according to claim 10, wherein the catalyst further comprises an alumina binder, the catalyst comprises 50-85 wt. % of a β zeolite, 0.5-5 wt. % of an active metal component in terms of metal element, 0-6.5 wt. % of an auxiliary metal component in terms of oxidation state, and 10-45 wt. % of an alumina binder, based on the total amount of the catalyst.

14. The method according to claim 13, wherein the catalyst comprises 60-85 wt. % of a β zeolite, 0.5-3 wt. % of an active metal component in terms of reduced state, 0.5-5.5 wt. % of an auxiliary metal component in terms of oxidation state, and 15-34.5 wt. % of an alumina binder, based on the total amount of the catalyst.

15. The method according to claim 10, wherein the contact reaction is performed under the circumstance without introducing ammonia gas.

16. The method according to claim 10, wherein the contact reaction is carried out intermittently, the temperature of the contact reaction is within the range of 130-180° C., the pressure of the contact reaction is within the range of 2-4 MPa, the reaction time is within the range of 4-8 h; or
the contact reaction is carried out continuously, the temperature of the contact reaction is within the range of 160-240° C., the pressure of the contact reaction is within the range of 1.5-3 MPa.

17. The method according to claim 10, wherein the aniline is reacted through a fixed bed reactor containing the catalyst;
and/or, the contact reaction is carried out intermittently, the used amount of the catalyst accounts for 1-4 wt. % of the used amount of the aniline;
and/or, the contact reaction is carried out continuously, the liquid hourly mass space velocity of the aniline is within the range of 0.5-3 h⁻¹.

18. The method according to claim 10, wherein the aniline is fed from the bottom of the fixed bed reactor and passes through a catalyst bed.

19. The method according to claim 9, wherein the aniline is fed from the bottom of the fixed bed reactor and passes through a catalyst bed.
